(19) United States
(12) Patent Application Publication
(10) Pub. No.: US 2025/0333567 A1
(43) Pub. Date: Oct. 30, 2025

(54) APPARATUS AND METHOD FOR PREPARING POLYLACTIC ACID MICROSPHERES

(71) Applicant: Hangzhou Singclean Medical Products Co., Ltd, Hangzhou (CN)

(72) Inventors: Jiaxiang Zhang, Hangzhou (CN); Ying Zhu, Hangzhou (CN); Qiulin He, Hangzhou (CN); Xue Tan, Hangzhou (CN); Wei Huang, Hangzhou (CN)

(21) Appl. No.: 19/014,264

(22) Filed: Jan. 9, 2025

(30) Foreign Application Priority Data
Apr. 30, 2024 (CN) 202410534098.2

(51) Int. Cl. C08J 3/16

(52) U.S. Cl. CPC C08J 3/16; C08J 2367/02

(57) ABSTRACT

Provided are an apparatus and a method for preparing polylactic acid microspheres. The apparatus includes a reactor body, a reactor cover, and a solvent distillation and recovery device. The method for preparing polylactic acid microspheres includes the following steps: adding a solution of polylactic acid in an organic solvent to an aqueous solution of polyvinyl alcohol; emulsifying, and heating the reaction liquid in real time, whereby the solvent in the droplets of polylactic acid microspheres is evaporated and the droplets are solidified into polylactic acid microspheres; and washing and drying the crude polylactic acid microspheres, to obtain finished polylactic acid microspheres. By using the apparatus and method for preparing polylactic acid microspheres according to the present invention, polylactic acid microspheres with concentrated particle size distribution is prepared, and the organic solvent is recovered and reused.

### CROSS-REFERENCE TO RELATED APPLICATION

The present application claims priority from Chinese Patent Application No. 202410534098.2 filed on Apr. 30, 2024, the contents of which are incorporated herein by reference in their entirety.

### TECHNICAL FIELD

The present invention relates to a medical beauty product, and specifically to an apparatus and a method for preparing polylactic acid microspheres.

### BACKGROUND

Biodegradable polymer microspheres represented by polylactic acid are widely used in the field of medical beauty because of their good biocompatibility and biodegradability. Lactic acid is an intermediate product in the degradation of polylactic acid, which can eventually be degraded into carbon dioxide and water harmless to human body and environment. During the degradation process, polylactic acid gradually releases lactic acid, enhances the activities of fibroblast transforming growth factor TGF-β, proline hydroxylase and matrix metalloproteinase inhibitor, stimulates the proliferation, differentiation and metabolism of fibroblasts, and promotes the secretion of collagen, until polylactic acid is completely degraded. Therefore, a collagen stimulating filler mainly made of polylactic acid microspheres can ensure a long-lasting filling effect.

The main preparation methods of polylactic acid microspheres include spray drying, phase separation, emulsification-solvent evaporation, and so on. By spraying a suspension or an emulsion into hot air, the spray drying can quickly evaporate the solvent and allow for the formation of solid particles in a few seconds, during which strict control of the temperature of hot air is required. The phase separation is conducted by making use of the physicochemical properties of a polymer, during which a non-solvent liquid is added to a polymer solution, to form a polymer precipitate. However, a large amount of organic solvent is consumed in the process. The emulsification-solvent evaporation has the advantages of convenient operation, simple process and easy expansion of production, and can be widely used. In the emulsification-solvent evaporation, the particle size is adjusted generally by adjusting the rotational speed for emulsification, the solvent type, and the emulsification temperature and time. However, if the process conditions are not well controlled, irregular morphology of the polylactic acid microspheres, agglomeration and adhesion of the microspheres, and wide size distribution of the microspheres will occur, seriously affecting the use of the microspheres.

In CN116139790A, polylactic acid microspheres with uniform particle size are prepared by using a microfluidic apparatus based on the principle of emulsification-solvent evaporation. However, the microfluidic apparatus has a high precision requirement, and the yield of microspheres is low, so the method has difficulty in wide use in industrial production. In CN115531607A, CN114931554A, and CN108653817A, the preparation of microspheres by emulsification-solvent evaporation is also reported, but the method for removing the organic solvent used to dissolve the polyester material is natural evaporation or evaporation by heating, causes great harm to the environment and production personnel. In the microsphere preparation apparatus and the microsphere preparation method disclosed in the present invention, a solvent distillation and recovery device is used to completely recover the organic solvent, thus greatly reducing the harm to the environment and production personnel. Accordingly, the present invention has remarkable inventiveness.

In addition to the above description, the present invention also has the following the beneficial effects. In the microsphere preparation method of the present invention, the volume ratio of the continuous phase to the dispersed phase, the weight ratio of polyvinyl alcohol to polylactic acid, the stirring speed, the rotational speed for emulsification, the removal temperature of the organic solvent, and others are controlled. In CN115531607A, only the mass fraction of surfactant, the stirring speed and shear rate for homogenization are controlled. In CN114931554A, the weight ratio of the degradable polymer to the surfactant is not mentioned. In the microsphere preparation method of the present invention, numerous parameters affecting the quality of finished polylactic acid microspheres are controlled, thus effectively ensuring the applicability of the finished polylactic acid microspheres.

### SUMMARY

In view of the problems existing in the prior art, improvements are made in the present invention. The present invention provides an apparatus and a method for preparing polylactic acid microspheres. The present invention is accomplished through the following technical solutions.

The present invention discloses an apparatus for preparing polylactic acid microspheres. The apparatus includes: a reactor body, a reactor cover provided on the reactor body to cover an opening of the reactor body, and a solvent recovery and distillation device connected to the reactor cover.

The reactor body includes an inner cylinder, an outer cylinder, an interlayer formed between the inner cylinder and the outer cylinder, an interlayer inlet and outlet provided on an outer surface of the interlayer, a tank bottom valve below the reactor body, and a high-temperature circulating device in communication with the interlayer inlet and outlet.

The reactor cover includes a feed inlet, a distillation port, a stirring device, and an emulsifying device provided on a surface of the reactor cover. The stirring device includes a stirring motor and a stirring rod and blade connected to the stirring motor. The stirring motor is located at the center on the top of the reactor cover, and the stirring rod and blade extend into the inner cylinder of the reactor body, to stir the reaction liquid. The emulsifying device includes an emulsifying motor located on the top of the reactor cover, and an emulsifying head connected to the emulsifying motor. The emulsifying head extends into the inner cylinder of the reactor body, with which the reaction liquid is sheared to produce droplets of polylactic acid microspheres.

The solvent distillation and recovery device includes a condensing tube, a circulating inlet and outlet provided on the surface of the condensing tube, a low-temperature circulating device in communication with the circulating inlet and outlet, a reflux head provided at the bottom of the condensing tube, and a collection flask connected below the reflux head.

As a further improvement, the high-temperature circulating device of the present invention circulates the circulating liquid of the high-temperature circulating device through the interlayer inlet and outlet, to supply heat to the reactor body in real time, whereby the solvent in the droplets of polylactic acid microspheres is evaporated and the droplets are solidified into polylactic acid microspheres. The tank bottom valve is used to discharge the reaction liquid in the inner cylinder.

As a further improvement, the reflux head of the present invention is provided with three open ends. A first end is connected to and in communication with the distillation port, a second end is connected to and in communication with the bottom of the condensing tube, and a third end is connected to and in communication with the collection flask, to achieve the guidance of the evaporated solvent to flow from the inner cylinder to the condensing tube and the collection of the condensed solvent. The collection flask is used to collect the condensed solvent. The condensing tube has a double-layer structure and is connected to the low-temperature circulating device to realize the condensation of the evaporated solvent.

As a further improvement, the reactor body of the present invention is connected to the reactor cover by a seal ring, a screw, or a buckle. The reactor body, the reactor cover, and the solvent distillation and recovery device are made of a corrosion-resistant material, and preferably 316 stainless steel.

The present invention further discloses a method for preparing polylactic acid microspheres, by using the apparatus for preparing polylactic acid microspheres. The method includes the following steps:
1) preparing a continuous-phase polyvinyl alcohol solution and a dispersed-phase polylactic acid solution respectively;
2) feeding the continuous-phase polyvinyl alcohol solution into the inner cylinder of the inner cylinder, and then slowly adding the dispersed-phase polylactic acid solution into the continuous-phase polyvinyl alcohol solution, to form a reaction liquid; and enabling the reaction liquid to flow by the stirring rod and blade driven by the stirring motor, and continuously shearing the reaction liquid by the emulsifying head driven by the emulsifying motor, to produce droplets of polylactic acid microspheres;
3) stopping the emulsifying motor, while the stirring motor is maintained to work, and heating the reaction solution in real time by the high-temperature circulating device in real time, whereby the solvent in the droplets of polylactic acid microspheres is evaporated and the droplets are solidified into polylactic acid microspheres; and cooling the evaporated solvent by the low-temperature circulating device in real time, and flowing the condensed solvent through the reflux head and collecting in the collection flask; and
4) obtaining a reaction liquid containing crude polylactic acid microspheres after the solvent is distilled, washing and drying the crude polylactic acid microspheres, to obtain finished polylactic acid microspheres.

As a further improvement, the continuous-phase polyvinyl alcohol solution in the present invention is a solution prepared by adding polyvinyl alcohol into water for injection, stirring and dissolving, and having a concentration of 0.05-0.1 g/ml. The dispersed-phase polylactic acid solution is a dispersed-phase solution prepared by adding polylactic acid into one or a mixture of dichloromethane and chloroform, stirring, and dissolving in a sealed container, and having a concentration of 0.1-0.2 g/ml.

As a further improvement, the volume ratio of the continuous phase to the dispersed phase is 5:1-50:1, the weight ratio of polyvinyl alcohol to polylactic acid is 1:0.1-1:2, the stirring speed is 30-150 rpm, and the rotational speed for emulsification is 100-3000 rpm.

As a further improvement, the volume ratio of the continuous phase to the dispersed phase is 8:1-20:1, the weight ratio of polyvinyl alcohol to polylactic acid is 1:0.3-1:0.7, the stirring speed is 60-90 rpm, and the rotational speed for emulsification is 500-1500 rpm.

As a further improvement, the solvent in the droplets of polylactic acid microspheres is removed by heating in Step 3) of the present invention, where the heating temperature is 30-50° C., and preferably 40-50° C.

The present invention further discloses use of polylactic acid microspheres prepared by the method for preparing polylactic acid microspheres. The polylactic acid microspheres, as a component of a filler, is suspended in an aqueous solution of sodium carboxymethyl cellulose and mannitol, and freeze-dried to obtain a polylactic acid filler for medical beauty injection.

The present invention has the following beneficial effects.
1. In the apparatus for preparing polylactic acid microspheres of the present invention, a stirring device and an emulsifying device are both provided, which are used with the method for preparing polylactic acid microspheres of the present invention to efficiently and uniformly disperse the dispersed-phase polylactic acid solution into polylactic acid droplets, whereby polylactic acid microspheres with concentrated particle size distribution can be rapidly prepared in large quantities.
2. Further, in the apparatus for preparing polylactic acid microspheres of the present invention, a circulating liquid heated by the high-temperature circulating device is flowed in the interlayer formed between the inner cylinder and the outer cylinder, thus realizing the water bath heating of the reaction liquid and avoiding the temperature fluctuation caused by directly heating the reaction liquid. By the combination with the method for preparing the microspheres of the present invention, the heating temperature is controlled to range from 30 to 50° C., whereby the temperature of polylactic acid in the reaction liquid does not exceed its glass transition temperature all the time, to avoid the change of the physical and chemical properties of the polylactic acid microspheres.
3. Further, in the apparatus for preparing polylactic acid microspheres of the present invention, when polylactic acid droplets are heated and solidified into polylactic acid microspheres, the evaporated dichloromethane or trichloromethane gas can be guided by the reflux head to flow to the condensing tube, where the evaporated solvent is continuously condensed. The condensed solvent flows through the reflux head and is collected in the collection flask, to realize the complete recovery and reuse of the organic solvent, avoid the toxic effect on production personnel and the damage to the environment, and also realize the reuse of solvents.
4. In the method for preparing microspheres of the present invention, the volume ratio of the continuous phase to the dispersed phase is 5:1-50:1, the weight ratio of polyvinyl alcohol to polylactic acid is 1:0.1-1:2, the stirring speed is 30-150 rpm, and the rotational speed for emulsification is 100-3000 rpm. By using the parameters in the above ranges, the prepared polylactic acid microspheres have good spheroidization effect, high yield, and concentrated particle size distribution.
5. In the method for preparing microspheres of the present invention, the volume ratio of the continuous phase to the dispersed phase is 8:1-20:1, the weight ratio of polyvinyl alcohol to polylactic acid is 1:0.3-1:0.7, the stirring speed is 60-90 rpm, and the rotational speed for emulsification is 500-1500 rpm. By using the parameters in the above ranges, the prepared polylactic acid microspheres have better spheroidization effect, higher yield, and more concentrated particle size distribution.
6. Further, the temperature for removing the solvent in the droplets of the polylactic acid microspheres is controlled to be 30-50° C. and preferably 40-50° C. in the present invention. The polylactic acid microspheres solidified in the temperature range has smoother surface, less impurities and less organic solvent residue.
7. The polylactic acid microspheres prepared in the present invention has high biocompatibility, can stimulate the regeneration of collagen, and, as a component of a filler, can be suspended in an aqueous solution of sodium carboxymethyl cellulose and mannitol, and used for medical beauty injection after freeze-drying.

### BRIEF DESCRIPTION OF THE DRAWINGS

FIG. 1 is a schematic diagram of one embodiment of an apparatus for preparing polylactic acid microspheres;

FIG. 2 shows the particle size distribution of polylactic acid microspheres prepared by a method for preparing polylactic acid microspheres;

FIG. 3 shows an image of polylactic acid microspheres with a particle size ranging from 10 to 50 μm prepared by a method for preparing polylactic acid microspheres;

FIG. 4 shows a tissue slice of collagen stimulated to be produced by polylactic acid microspheres prepared in the present invention, 3 months after implantation under rabbit skin;

FIG. 5 shows a tissue slice of collagen stimulated to be produced by polylactic acid microspheres prepared in the present invention, 6 months after implantation under rabbit skin;

### List of Reference Numerals

11 inner cylinder, 12 outer cylinder, 13 interlayer inlet and outlet, 14 tank bottom valve, 15 high-temperature circulating device;
21 feed inlet, 22 distillation port, 23 emulsifying motor, 24 emulsifying head, 25 stirring motor, 26 stirring rod and blade;
31 condensing tube, 32 condensing and circulating inlet and outlet, 33 low-temperature circulating device, 34 reflux head, 35 collection flask.

### DETAILED DESCRIPTION

The present invention will be further described below in connection with specific examples with reference to accompanying drawings. In the following description, more details are provided for well understanding of the present invention. However, it is obvious that the present invention can be implemented in many other ways different from the description, and similar expansion and deduction can be made by those skilled in the art without departing from the spirit of the present invention. Therefore, the protection scope of the present invention should not be limited by the description of the specific embodiments herein.

It is to be noted that these and other subsequent drawings are merely illustrative, which are not drawn to scale, and should not be taken as limitations on the actual scope of protection of the present invention.

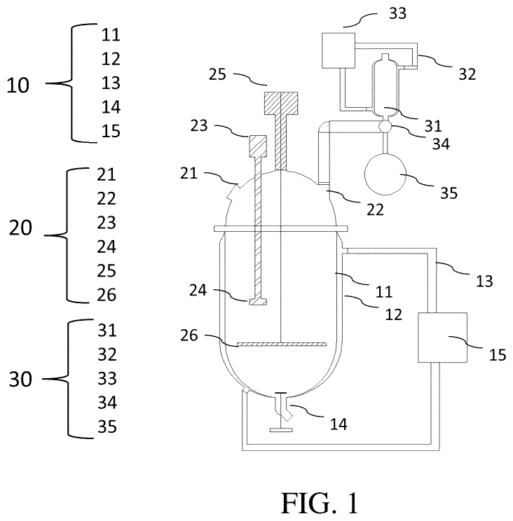

As shown in FIG. 1, the apparatus for preparing polylactic acid microspheres includes a reactor body 10, a reactor cover 20 provided on the reactor body to cover an opening of the reactor body, and a solvent recovery and distillation device 30 connected to the reactor cover.

As shown in FIG. 1, the reactor body 10 includes an inner cylinder 11, an outer cylinder 12, an interlayer 13 formed between the inner cylinder and the outer cylinder, an interlayer inlet and outlet 14 provided on an outer surface of the interlayer, a tank bottom valve 15 below the reactor body, and a high-temperature circulating device 15 in communication with the interlayer inlet and outlet.

As shown in FIG. 1, the reactor cover 20 includes a feed inlet 21, a distillation port 22, a stirring device, and an emulsifying device provided on a surface of the reactor cover. The stirring device includes a stirring motor 23 and a stirring rod and blade 24 connected to the stirring motor. The stirring motor 23 is located at the center on the top of the reactor cover, and the stirring rod and blade 24 extend into the inner cylinder 11 of the reactor body 10, to stir the reaction liquid. The emulsifying device includes an emulsifying motor 25 located on the top of the reactor cover, and an emulsifying head 26 connected to the emulsifying motor. The emulsifying head 26 extends into the inner cylinder of the reactor body 10, with which the reaction liquid is sheared to produce droplets of polylactic acid microspheres.

As shown in FIG. 1, the solvent distillation and recovery device 30 includes a condensing tube 31, a circulating inlet and outlet 32 provided on the surface of the condensing tube, a low-temperature circulating device 33 in communication with the circulating inlet and outlet, a reflux head 34 provided at the bottom of the condensing tube, and a collection flask 35 connected below the reflux head.

In one or more embodiments, the high-temperature circulating device 15 circulates the circulating liquid of the high-temperature circulating device through the interlayer inlet and outlet 13, to supply heat to the reactor body 10 in real time, whereby the solvent in the droplets of polylactic acid microspheres is evaporated and the droplets are solidified into polylactic acid microspheres. The tank bottom valve 14 is used to discharge the reaction liquid in the inner cylinder 11.

In one or more embodiments, the reflux head 34 is provided with three open ends. A first end is connected to and in communication with the distillation port 22, a second end is connected to and in communication with the bottom of the condensing tube 31, and a third end is connected to and in communication with the collection flask 35, to achieve the guidance of the evaporated solvent to flow from the inner cylinder 11 to the condensing tube 31 and the collection of the condensed solvent. The collection flask 35 is used to collect the condensed solvent. The condensing tube 31 has a double-layer structure and is connected to the low-temperature circulating device 33 to realize the condensation of the evaporated solvent.

In one or more embodiments, the reactor body 10 is connected to the reactor cover 20 by a seal ring, a screw, or a buckle. The reactor body 10, the reactor cover 20, and the solvent distillation and recovery device 30 are made of a corrosion-resistant material, and preferably 316 stainless steel.

In one or more embodiments, the dispersed phase is continuously and evenly added into the flowing phase through the feed inlet 21 at low rate, to improve the uniformity of the prepared polylactic acid microspheres.

In one or more embodiments, the continuous phase is an aqueous system containing polyvinyl alcohol surfactant; and the dispersed phase is an organic solvent system containing polylactic acid. The organic solvent is preferably one or a mixture of dichloromethane and chloroform. Due to the corrosiveness, flammability, and explosiveness of the organic solvent, the reactor is preferably an explosion-proof apparatus, preferably made of 316 stainless steel.

Through the description of the apparatus for preparing polylactic acid microspheres, a method for preparing polylactic acid microspheres with concentrated particle size distribution can be obtained. The preparation method mainly includes the following steps:
preparing a continuous-phase polyvinyl alcohol solution and a dispersed-phase polylactic acid solution respectively; feeding the continuous-phase polyvinyl alcohol solution into the inner cylinder of the inner cylinder 11, and then slowly adding the dispersed-phase polylactic acid solution into the continuous-phase polyvinyl alcohol solution, to form a reaction liquid; enabling the reaction liquid to flow by the stirring rod and blade 26 by the stirring motor 25, and continuously shearing the reaction liquid by the emulsifying head 24 driven by the emulsifying motor 23, to produce droplets of polylactic acid microspheres; stopping the emulsifying motor 34, while the stirring motor 25 is maintained to work, and heating the reaction solution in real time by the high-temperature circulating device 15 in real time, whereby the solvent in the droplets of polylactic acid microspheres is evaporated and the droplets are solidified into polylactic acid microspheres; cooling the evaporated solvent by the low-temperature circulating device 33 in real time, and flowing the condensed solvent through the reflux head 34 and collecting in the collection flask 35; and obtaining a reaction liquid containing crude polylactic acid microspheres after the solvent is distilled, washing and drying the crude polylactic acid microspheres, to obtain finished polylactic acid microspheres.

In one or more embodiments, the continuous-phase polyvinyl alcohol solution is a solution prepared by adding polyvinyl alcohol into water for injection, stirring and dissolving, and having a concentration of 0.05-0.1 g/ml. The dispersed-phase polylactic acid solution is a dispersed-phase solution prepared by adding polylactic acid into one or a mixture of dichloromethane and chloroform, stirring, and dissolving in a sealed container, and having a concentration of 0.1-0.2 g/ml.

In one or more embodiments, the volume ratio of the continuous phase to the dispersed phase is 5:1-50:1, the weight ratio of polyvinyl alcohol to polylactic acid is 1:0.1-1:2, the stirring speed is 30-150 rpm, and the rotational speed for emulsification is 100-3000 rpm.

In one or more embodiments, the volume ratio of the continuous phase to the dispersed phase is 8:1-20:1, the weight ratio of polyvinyl alcohol to polylactic acid is 1:0.3-1:0.7, the stirring speed is 60-90 rpm, and the rotational speed for emulsification is 500-1500 rpm.

In one or more embodiments, the solvent in the droplets of polylactic acid microspheres is removed by heating in the method for preparing polylactic acid microspheres, where the heating temperature is 30-50° C., and preferably 40-50° C.

The method for preparing polylactic acid microspheres is further described by way of specific examples.

### Example 1

(1) Poly (L-lactic acid) was dissolved in dichloromethane to prepare a dispersed-phase solution with a concentration of 0.5 g/ml. The solid was dissolved by stirring in a sealed container, to prevent the volatilization of dichloromethane. In this way, a dispersed phase was obtained. Polyvinyl alcohol was added to purified water to prepare a continuous-phase solution with a concentration of 0.06 g/ml.
(2) The continuous phase was added into the inner cylinder in such a volume that the continuous phase submerges the stirring rod and blade and the emulsifying head. The stirring motor was started, and the stirring speed was set to 50 rpm. The emulsifying motor was started, and the rotational speed was set to 750 rpm. The dispersed phase was continuously added to the continuous phase at a low rate, where the final ratio of the continuous phase to the dispersed phase was 10:1. The emulsification was continued for 30 min.
(3) After emulsification, the emulsifying motor was stopped. The high-temperature circulating device was started to heat the reaction liquid, where the temperature was set at 40° C., to evaporate the solvent in the droplets of polylactic acid microspheres and solidify the droplets into polylactic acid microspheres. The evaporated solvent was recovered by the solvent distillation and recovery device.
(4) The solidified microspheres were collected, washed, and dried to obtain finished polylactic acid microspheres.

### Determination of Microsphere Properties:

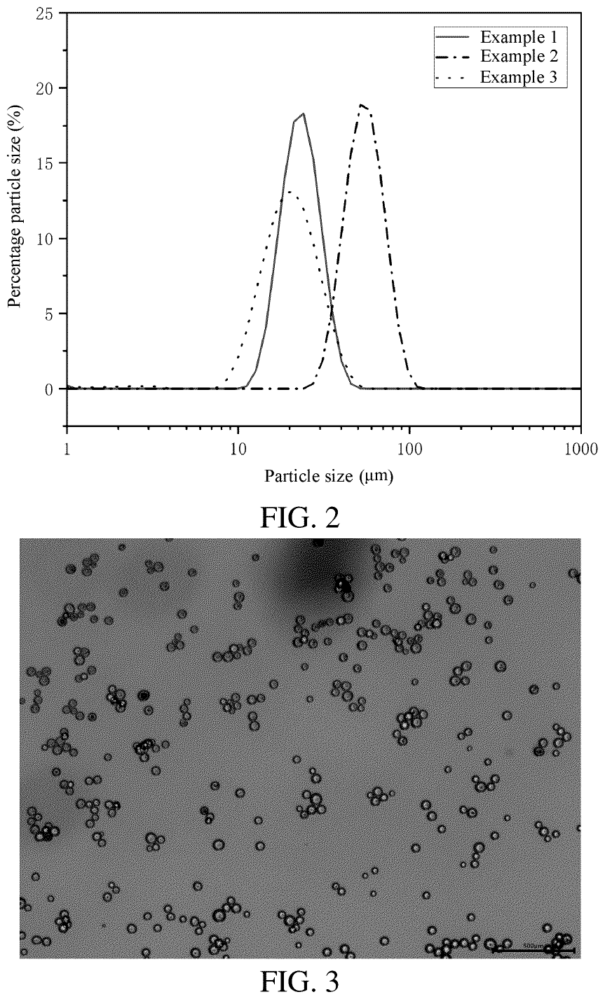

As shown in FIG. 3, the microspheres obtained in this example are regular spheres under an optical microscope. The microspheres obtained in this example were measured by a dry method using Mastersize3000 laser particle size analyzer. As shown in FIG. 2, the particle size distribution range of the microspheres is 10 to 50 μm. The proportion of microspheres with a particle size in the range of 15-45 μm is over 90%. The residual amount of dichloromethane is less than 0.01%, and the residual amount of polyvinyl alcohol is less than 0.5%.

### Example 2

Compared with Example 1, the concentration of the dispersed-phase solution was 0.2 g/ml.

### Determination of Microsphere Properties:

The microspheres obtained in this example are regular spheres under an optical microscope. The microspheres obtained in this example were measured by a dry method using Mastersize3000 laser particle size analyzer. As shown in FIG. 2, the particle size distribution range of the microspheres is 25 to 110 μm. The proportion of microspheres with a particle size in the range of 40-80 μm is over 90%. The residual amount of dichloromethane is less than 0.01%, and the residual amount of polyvinyl alcohol is less than 0.5%.

### Example 3

Compared with Example 1, the rotational speed for emulsification was 1000 rpm.

### Determination of Microsphere Properties:

The microspheres obtained in this example are regular spheres under an optical microscope. The microspheres obtained in this example were measured by a dry method using Mastersize3000 laser particle size analyzer. As shown in FIG. 2, the particle size distribution range of the microspheres is 10 to 40 μm. The proportion of microspheres with a particle size in the range of 15-30 μm is over 90%. The residual amount of dichloromethane is less than 0.01%, and the residual amount of polyvinyl alcohol is less than 0.5%.

Through the preparation method and examples of polylactic acid microspheres, polylactic acid microspheres with concentrated particle size distribution, smooth surface, and low residue can be obtained.

### Comparative Example 1

Compared with Example 1, various volume ratios of the continuous phase and the dispersed phase were set. The test results are shown in Table 1 below:

TABLE 1

| Continuous | Average particle | |
|---|---|---|
| phase:dispersed phase | size (μm) | Yield (%) |
| 5:1 | 68.3 | 87 |
| 8:1 | 28.5 | 85 |
| 10:1 | 24.3 | 82 |
| 20:1 | 26.8 | 60 |
| 30:1 | 28.9 | 43 |

As can be seen from Table 1, with the increasing volume ratio of the continuous phase to the dispersed phase, the average particle size decreases and then increases, but the yield decreases constantly. Therefore, the volume ratio of the continuous phase to the dispersed phase is appropriately from 8:1 to 20:1.

### Comparative Example 2

Compared with Example 1, various temperatures for distilling the solvent were set. The test results are shown in Table 2 below:

TABLE 2

| Solvent distillation | Distillation | Percentage of solvent |
|---|---|---|
| temperature (° C.) | time (h) | distilled (%) |
| 30 | 14 | 42 |
| 35 | 13.5 | 55 |
| 40 | 8 | 68 |
| 45 | 6.5 | 70 |
| 50 | 5 | 76 |

As can be seen from Table 2, with the increase of the solvent distillation temperature, the distillation time decreases and the percentage of solvent distilled decreases. Considering the glass transition temperature of polylactic acid of about 60° C., the distillation time and the percentage of solvent distilled, the heating temperature for solvent removal is appropriately in the range of 40 to 50° C.

### Example 4

The polylactic acid microspheres prepared in Example 3 were suspended in an aqueous solution of sodium carboxymethyl cellulose and mannitol, and then injected subcutaneously into rabbits. They animals were sacrificed respectively after 3 months and 6 months. The tissues at the injection site were removed, embedded with paraffin into tissue sections, and stained with Masson. The collagen regeneration was observed.

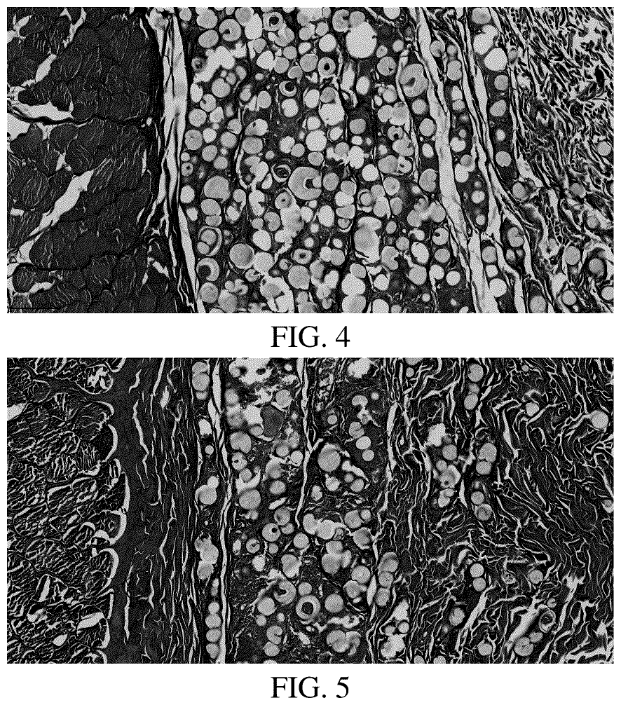

As shown in FIG. 4, round granular polylactic acid microspheres can be observed under the dermis, and occasionally a small number of polylactic acid microspheres are damaged. At the deep dermal implant of skin, the collagen fibers are arranged in disorder, and the network structure disappears. A small amount of fine new collagen is generated, which wraps the polylactic acid microspheres and divides them into several lobules. As shown in FIG. 5, round granular polylactic acid microspheres can be observed under the dermis, and the damaged filler increases slightly. At the deep dermal implant of skin, the collagen fibers are arranged in disorder, and the network structure disappears. More fine new collagen is produced, some collagen becomes large, and wraps the polylactic acid microspheres.

Although preferred embodiments of the present invention are disclosed, the present disclosure is not limited thereto. Possible changes and modifications can be made by any person skilled in the art without departing from the spirit and scope of the present invention. Therefore, any variations, equivalent changes and modifications made to the above embodiments according to the technical essence of the present invention without departing from the technical solution of the present invention shall fall within the scope of protection defined by the claims of the present invention.

What is claimed is:

1. An apparatus for preparing polylactic acid microspheres, comprising: a reactor body, a reactor cover provided on the reactor body to cover an opening of the reactor body, and a solvent recovery and distillation device connected to the reactor cover, wherein
the reactor body comprises an inner cylinder, an outer cylinder, an interlayer formed between the inner cylinder and the outer cylinder, an interlayer inlet and outlet provided on an outer surface of the interlayer, a tank bottom valve below the reactor body, and a high-temperature circulating device in communication with the interlayer inlet and outlet;
the reactor cover comprises a feed inlet, a distillation port, a stirring device, and an emulsifying device provided on a surface of the reactor cover; the stirring device comprises a stirring motor and a stirring rod and blade connected to the stirring motor, wherein the stirring motor is located at the center on the top of the reactor cover, and the stirring rod and blade extend into the inner cylinder of the reactor body, to stir the reaction liquid; the emulsifying apparatus comprises an emulsifying motor located on the top of the reactor cover, and an emulsifying head connected to the emulsifying motor, wherein the emulsifying head extends into the inner cylinder of the reactor body, with which the reaction liquid is sheared to produce droplets of polylactic acid microspheres; and
the solvent distillation and recovery device comprises a condensing tube, a circulating inlet and outlet provided on the surface of the condensing tube, a low-temperature circulating device in communication with the circulating inlet and outlet, a reflux head provided at the bottom of the condensing tube, and a collection flask connected below the reflux head.

2. The apparatus for preparing polylactic acid microspheres according to claim 1, wherein the high-temperature circulating device circulates the circulating liquid of the high-temperature circulating device through the interlayer inlet and outlet, to supply heat to the reactor body in real time, whereby the solvent in the droplets of polylactic acid microspheres is evaporated and the droplets are solidified into polylactic acid microspheres; and the tank bottom valve is used to discharge the reaction liquid in the inner cylinder.

3. The apparatus for preparing polylactic acid microspheres according to claim 2, wherein the reflux head is provided with three open ends, wherein a first end is connected to and in communication with the distillation port, a second end is connected to and in communication with the bottom of the condensing tube, and a third end is connected to and in communication with the collection flask, to achieve the guidance of the evaporated solvent to flow from the inner cylinder to the condensing tube and the collection of the condensed solvent; the collection flask is used to collect the condensed solvent; and the condensing tube has a double-layer structure and is connected to the low-temperature circulating device to realize the condensation of the evaporated solvent.

4. The apparatus for preparing polylactic acid microspheres according to claim 1, wherein the reactor body is connected to the reactor cover by a seal ring, a screw, or a buckle, and the reactor body, the reactor cover, and the solvent distillation and recovery device are made of a corrosion-resistant material.

5. A method for preparing polylactic acid microspheres by using the apparatus for preparing polylactic acid microspheres according to claim 1, the method comprising the following steps:

1. preparing a continuous-phase polyvinyl alcohol solution and a dispersed-phase polylactic acid solution respectively;

2. feeding the continuous-phase polyvinyl alcohol solution into the inner cylinder of the inner cylinder, and then slowly adding the dispersed-phase polylactic acid solution into the continuous-phase polyvinyl alcohol solution, to form a reaction liquid; enabling the reaction liquid to flow by the stirring rod and blade driven by the stirring motor, and continuously shearing the reaction liquid by the emulsifying head driven by the emulsifying motor, to produce droplets of polylactic acid microspheres;

3. stopping the emulsifying motor, while the stirring motor is maintained to work, and heating the reaction solution in real time by the high-temperature circulating device in real time, whereby the solvent in the droplets of polylactic acid microspheres is evaporated and solidify the droplets into polylactic acid microspheres; and cooling the evaporated solvent by the low-temperature circulating device in real time, and flowing the condensed solvent through the reflux head and collecting in the collection flask; and

4. obtaining a reaction liquid containing crude polylactic acid microspheres after the solvent is distilled, washing and drying the crude polylactic acid microspheres, to obtain finished polylactic acid microspheres.

6. The method for preparing polylactic acid microspheres according to claim 5, wherein the continuous-phase polyvinyl alcohol solution is a solution prepared by adding polyvinyl alcohol into water for injection, stirring and dissolving, and having a concentration of 0.05-0.1 g/ml; the dispersed-phase polylactic acid solution is a dispersed-phase solution prepared by adding polylactic acid into one or a mixture of dichloromethane and chloroform, stirring, and dissolving in a sealed container, and having a concentration of 0.1-0.2 g/ml.

7. The method for preparing polylactic acid microspheres according to claim 5, wherein the volume ratio of the continuous phase to the dispersed phase is 5:1-50:1, the weight ratio of polyvinyl alcohol to polylactic acid is 1:0.1-1:2, the stirring speed is 30-150 rpm, and the rotational speed for emulsification is 100-3000 rpm.

8. The method for preparing polylactic acid microspheres according to claim 7, wherein the volume ratio of the continuous phase to the dispersed phase is 8:1-20:1, the weight ratio of polyvinyl alcohol to polylactic acid is 1:0.3-1:0.7, the stirring speed is 60-90 rpm, and the rotational speed for emulsification is 500-1500 rpm.

9. The method for preparing polylactic acid microspheres according to claim 5, wherein the solvent in the droplets of polylactic acid microspheres is removed by heating in Step 3), wherein the heating temperature is 30-50° C.

10. Use of the polylactic acid microspheres prepared by the method for preparing polylactic acid microspheres according to claim 5, wherein the polylactic acid microspheres, as a component of a filler, is suspended in an aqueous solution of sodium carboxymethyl cellulose and mannitol, and freeze-dried to obtain a polylactic acid filler for medical beauty injection.

11. The method for preparing polylactic acid microspheres according to claim 6, wherein the volume ratio of the continuous phase to the dispersed phase is 5:1-50:1, the weight ratio of polyvinyl alcohol to polylactic acid is 1:0.1-1:2, the stirring speed is 30-150 rpm, and the rotational speed for emulsification is 100-3000 rpm.

12. The method for preparing polylactic acid microspheres according to claim 8, wherein the solvent in the droplets of polylactic acid microspheres is removed by heating in Step 3), wherein the heating temperature is 30-50° C.

13. A method for preparing polylactic acid microspheres by using the apparatus for preparing polylactic acid microspheres according to claim 2, the method comprising the following steps:
1) preparing a continuous-phase polyvinyl alcohol solution and a dispersed-phase polylactic acid solution respectively;
2) feeding the continuous-phase polyvinyl alcohol solution into the inner cylinder of the inner cylinder, and then slowly adding the dispersed-phase polylactic acid solution into the continuous-phase polyvinyl alcohol solution, to form a reaction liquid; enabling the reaction liquid to flow by the stirring rod and blade driven by the stirring motor, and continuously shearing the reaction liquid by the emulsifying head driven by the emulsifying motor, to produce droplets of polylactic acid microspheres;
3) stopping the emulsifying motor, while the stirring motor is maintained to work, and heating the reaction solution in real time by the high-temperature circulating device in real time, whereby the solvent in the droplets of polylactic acid microspheres is evaporated and solidify the droplets into polylactic acid microspheres; and cooling the evaporated solvent by the low-temperature circulating device in real time, and flowing the condensed solvent through the reflux head and collecting in the collection flask; and
4) obtaining a reaction liquid containing crude polylactic acid microspheres after the solvent is distilled, washing and drying the crude polylactic acid microspheres, to obtain finished polylactic acid microspheres.

14. A method for preparing polylactic acid microspheres by using the apparatus for preparing polylactic acid microspheres according to claim 3, the method comprising the following steps:
1) preparing a continuous-phase polyvinyl alcohol solution and a dispersed-phase polylactic acid solution respectively;
2) feeding the continuous-phase polyvinyl alcohol solution into the inner cylinder of the inner cylinder, and then slowly adding the dispersed-phase polylactic acid solution into the continuous-phase polyvinyl alcohol solution, to form a reaction liquid; enabling the reaction liquid to flow by the stirring rod and blade driven by the stirring motor, and continuously shearing the reaction liquid by the emulsifying head driven by the emulsifying motor, to produce droplets of polylactic acid microspheres;
3) stopping the emulsifying motor, while the stirring motor is maintained to work, and heating the reaction solution in real time by the high-temperature circulating device in real time, whereby the solvent in the droplets of polylactic acid microspheres is evaporated and solidify the droplets into polylactic acid microspheres; and cooling the evaporated solvent by the low-temperature circulating device in real time, and flowing the condensed solvent through the reflux head and collecting in the collection flask; and
4) obtaining a reaction liquid containing crude polylactic acid microspheres after the solvent is distilled, washing and drying the crude polylactic acid microspheres, to obtain finished polylactic acid microspheres.

15. A method for preparing polylactic acid microspheres by using the apparatus for preparing polylactic acid microspheres according to claim 4, the method comprising the following steps:
1) preparing a continuous-phase polyvinyl alcohol solution and a dispersed-phase polylactic acid solution respectively;
2) feeding the continuous-phase polyvinyl alcohol solution into the inner cylinder of the inner cylinder, and then slowly adding the dispersed-phase polylactic acid solution into the continuous-phase polyvinyl alcohol solution, to form a reaction liquid; enabling the reaction liquid to flow by the stirring rod and blade driven by the stirring motor, and continuously shearing the reaction liquid by the emulsifying head driven by the emulsifying motor, to produce droplets of polylactic acid microspheres;
3) stopping the emulsifying motor, while the stirring motor is maintained to work, and heating the reaction solution in real time by the high-temperature circulating device in real time, whereby the solvent in the droplets of polylactic acid microspheres is evaporated and solidify the droplets into polylactic acid microspheres; and cooling the evaporated solvent by the low-temperature circulating device in real time, and flowing the condensed solvent through the reflux head and collecting in the collection flask; and
4) obtaining a reaction liquid containing crude polylactic acid microspheres after the solvent is distilled, washing and drying the crude polylactic acid microspheres, to obtain finished polylactic acid microspheres.
